(19) United States
(12) Patent Application Publication
(10) Pub. No.: US 2026/0232352 A1
(43) Pub. Date: Aug. 13, 2026

(54) SURGICAL INSTRUMENT PORT FOR VAGINAL NATURAL ORIFICE TRANSLUMINAL ENDOSCOPIC SURGERY

(71) Applicant: KOC UNIVERSITES, Istanbul (TR)

(72) Inventors: Serdar AYDIN, Istanbul (TR); Mert YASLI, Istanbul (TR)

(73) Assignee: KOC UNIVERSITES, Istanbul (TR)

(21) Appl. No.: 19/470,838

(22) PCT Filed: Mar. 11, 2024
(86) PCT No.: PCT/TR2024/050240

§ 371 Date: Sep. 30, 2025

(30) Foreign Application Priority Data
Apr. 4, 2023 (TR) 2023/003681

(51) Int. Cl. A61B 17/42; A61B 1/00; A61B 17/00; A61B 17/34

(52) U.S. Cl. CPC A61B 17/42; A61B 1/00154; A61B 17/00234; A61B 17/3423; A61B 2017/00238; A61B 2017/00526; A61B 2017/00862; A61B 2017/3433; A61B 2017/3445; A61B 2017/345

(57) ABSTRACT

A surgical instrument port is designed for use in vaginal natural orifice transluminal endoscopic surgery, made of a disposable, biocompatible and elastic material, and allows multiple surgical instruments and endoscopes to simultaneously have access to the desired body cavity.

### FIELD OF THE INVENTION

The present invention relates to a disposable, biocompatible and elastic surgical instrument port designed for use in vaginal natural orifice transluminal endoscopic surgery.

### BACKGROUND OF THE INVENTION

Natural Orifice Transluminal Endoscopic Surgery (NOTES) is a technique which has emerged from the need to reduce surgical morbidity caused by the surgical incision. Instead, the lumens of the gastrointestinal or genitourinary system are used as the ports which allow access to the abdominal cavity through mouth, anus, vagina, or urethra. By reducing the need to make incisions on the abdominal wall, it is aimed to provide easier convalescence for patients and the cosmetic outcomes of the surgery are minimized [1].

Vaginal NOTES (vNOTES) is an approach which involves making incision through the posterior vaginal fornix into the pouch of Douglas located between the uterus and colon and inserting a transvaginal surgical port including endoscopic surgical instruments for the specific procedure. In some cases that require removal of the anterior uterine myoma, the anterior fornix may also be dissected [2, 3]. vNOTES is considered to be the safest option among other NOTES techniques due to easier closure of culdotomy instead of entry through stomach. Furthermore, the vagina can be easily decontaminated, which reduces the risk of postoperative infection [1, 5]. There is no port designed specifically for this surgery. Alternatively, ports used in transabdominal surgery are adapted to transvaginal surgery. The common feature of these ports is that they have multiple holes for the insertion of different instruments and allow multiple instruments to pass through a single hole with the advantage of reducing the number of incisions [4].

vNOTES is used in a variety of different types of surgical operation. The technique was first used in a cholecystectomy case, followed by appendectomy, nephrectomy and gynecologic cases [1]. The type of gynecologic surgery in which the technique is most commonly used has been the removal uterus (hysterectomy) by 59% [5]. In a study by Wand et al., it has been found that vNOTES is equivalent to laparoscopic surgery for sentinel lymph node mapping in hysterectomy; however, it has a lower complication rate due to the surgical procedure [6]. Myomectomy is also an important field of application of this technique, and it has been reported that even the required operation time is reduced [2, 3].

The process of adapting existing devices to vNOTES, which can be considered as a novel method, poses an important problem. Surgical ports used in transabdominal surgery are not completely suitable for transvaginal surgery and are adapted to transvaginal surgery for each surgical procedure with the help of certain modifications without being standardized. The lack of standardization raises questions about the safety of surgical ports for patients and surgery. At this point, it is aimed to facilitate the use of the surgical port of the present invention that we have designed, by manufacturing it only with customization to individual patient, and in addition to this, by making the port standardized.

The vNOTES technique requires 1 endoscope and 2 surgical instruments to access the target tissue. When working in a small surgical site, it advantageous to insert these instruments through a single port in order to increase their control and efficiency in the tissue. In addition, every surgical incision may cause complications during the recovery period. Therefore, it is envisaged to use surgical port and insert multiple surgical instruments through a single-incision by reducing the number of incisions. Due to the nature of the technique, carbon dioxide must be present in the surgical site at constant pressure and volume. Thus, the necessary space for surgery is created, clear endoscopic visibility is ensured and the removed tissues can be moved away from the surgical site. The lack of a port for this surgery poses a problem with regard to these issues. The ports adapted cannot provide gas tightness standardly. In addition, ports that will be manufactured without taking into account the patient's measurements may also cause problems in terms of gas tightness.

There are similar applications developed in the literature against the technical problems mentioned and identified above. A method, which has been developed at Ege University, Faculty of Medicine by using one circular silicone pessary, powder-free surgical glove and trocar instruments, is based on creating a surgical port before each procedure with the principle of using glove fingers as a pathway through which surgical instruments pass. The tip of the glove fingers is pierced with the help of a trocar, a silicone pessary is placed on its rubber part on the wrist and fixed. Trocars are fixed to the glove fingers by means of 3-4 mm long pieces cut from the other glove, and thus an air tightness is obtained. Then, the instrument created is fixed to the vaginal wall with the pressure created by the pessary and made ready for use. This method reduces the cost of the surgical procedure and saves time by being produced within 2 minutes [7]. It can be said that the disadvantage of this method is that the port created is non-durable and too flexible. Therefore, it is difficult to ensure the necessary stability for the passage of the instruments.

Another method found is called laparoendoscopic single-site surgery (LESS) [8-11]. In this method, multiple surgical instruments enter the biocompatible material made of plastic through different ports and access to the surgical site through a single port. Different variations of this technique are described in the studies. It can be said that their common features are the expansion of the colpotomy with blunt dissection after incision of the posterior vaginal fornix and placement of a single port on the colpotomy line. This method is more durable in terms of stability than glove ports; however, it is more costly. Also, unlike gloves, the materials should be sterilized before surgery.

In U.S. Pat. No. 8,267,858 B2, an application known in the state of the art, although the GelPort method is similar to LESS, it is provided as a commercialized and standardized version of it. This device, which is used by being adapted from transabdominal surgical ports, is adapted to transvaginal surgery, thereby creating a field of application. EP2814432 B1, U.S. Pat. Nos. 8,608,652 B2 and U.S. 9,521,996 B2 have also looked for solutions to similar technical problems.

### SUMMARY OF THE INVENTION

The objective of the present invention is to realize a transvaginal surgical instrument port in order to ensure that endoscopic surgical instruments used for vaginal natural orifice transluminal endoscopic surgery have access to surgical site through a single entry point, the extracted tissues are removed from the surgical site, and air insulation is provided to keep the carbon dioxide gas to be used in surgery at a constant volume in the desired body cavity.

A further objective of the present invention is to realize a customized transvaginal surgical instrument port, which is standardized according to vNOTES surgery for the safety of the patient and the surgical procedure. Thus, ease of use is also ensured. Another objective of the present invention is to increase the success of the surgery by reducing the possibility of injury during surgical operation by means of the transvaginal surgical instrument port designed for use in surgical procedure.

A further objective of the present invention is to minimize the surgical and incisional complications of vNOTES technique and reduce the risk of postoperative infection.

Another objective of the present invention is to realize a cost-efficient and ergonomic transvaginal surgical instrument port.

### BRIEF DESCRIPTION OF THE DRAWINGS

“Surgical Instrument Port for Vaginal Natural Orifice Transluminal Endoscopic Surgery”, which is realized to fulfill the objective of the present invention, is illustrated in the accompanying figures, in which:

FIG. 1. is the isometric views (a-b) of the working principle of the port and plugs.

FIG. 2. is a 2D side-view of the port.

FIG. 3. is a view of the middle portion (horizontal section in the vaginal direction).

FIG. 4. is a view of the port from another angle.

FIG. 5. is a vertical cross-sectional view of the port.

FIG. 6. is an isometric top view of the port.

FIG. 7. is the views (a-e) of the plug from different angles.

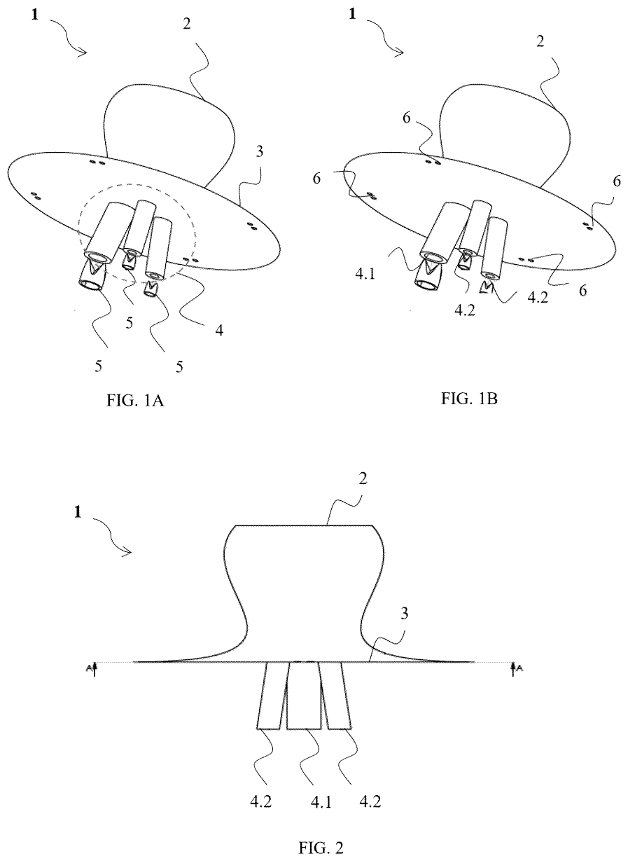

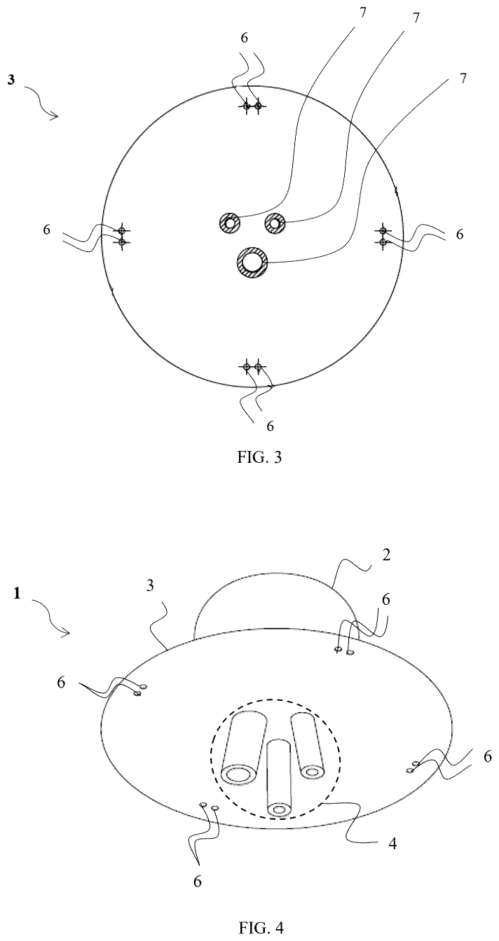

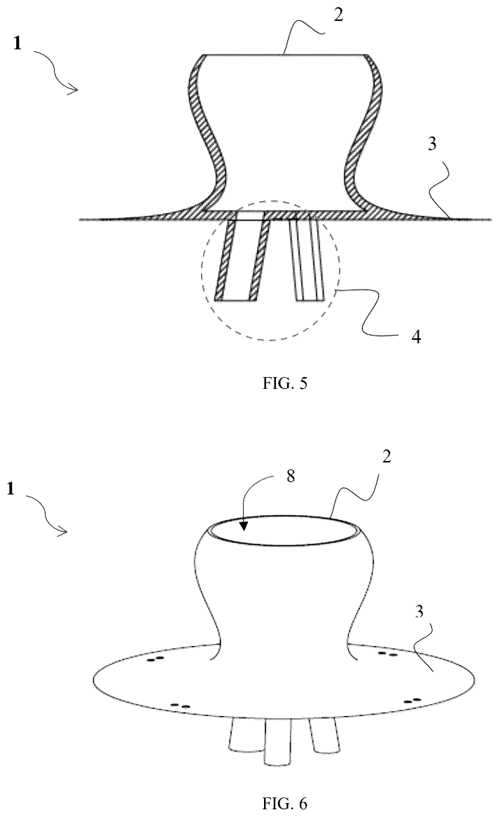

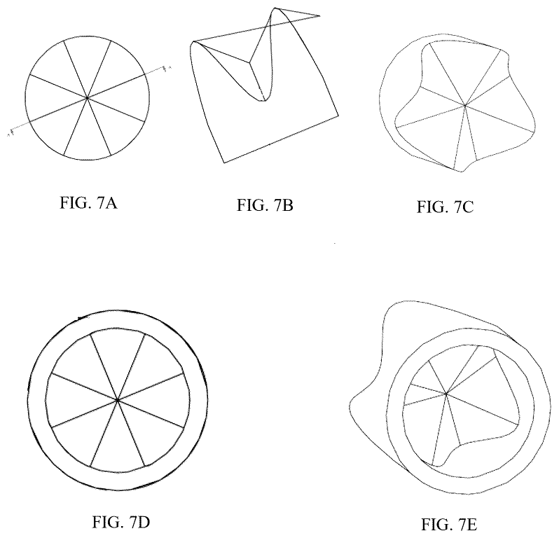

### DETAILED DESCRIPTION OF THE INVENTION

The present invention relates to a transvaginal surgical instrument port, which overcomes all the above-mentioned problems in the state of the art and provides additional advantages to the related field of the art. The present invention is a transvaginal surgical (NOTES) instrument port, which is designed and developed for use in vaginal natural orifice transluminal endoscopic surgery (vNOTES). This is the first port designed specifically for this surgery. Endoscopic surgical instruments, which will be used during the surgical procedure, are inserted into this port and used in the procedure. The present invention can also be manufactured according to the patient's measurements.

For the convenience of the reader, the numbers of the parts, which are shown in the drawings with brief description above and referred to in the specification, are given below.
1. Surgical Instrument Port
2. Lower portion (2)
3. Middle portion (3)
4. Passage
4.1. Passage with a diameter/width of 10 mm
4.2. Passage with a diameter/width of 5 mm
5. Plug (3 plugs in total in dimensions compatible with the passages, namely one plug of 10 mm and the other two of 5 mm)
6. Hole for the suture thread (8 holes in total, in groups of two)
7. Hole for the passage (3 holes in total in dimensions compatible with the passages, namely one hole with a diameter of 10 mm and the other two with a diameter of 5 mm)
8. Opening (on the lower portion)
   (The “A↑” designation in FIG. 2 indicates the direction towards the cavity.)

The present invention relates to a transvaginal surgical instrument port (1) used in the applications of vaginal natural orifice transluminal endoscopic surgery.

The main body of the present invention comprises a lower portion (2) (2) which enters into the cavity (groove; vagina), a thin middle portion (3) (3) which increases the air tightness and allows surgical instruments to enter into the cavity, and 3 surgical instrument insertion passages (4) (“passages”) which remain on the outermost part.

The present invention essentially comprises:
a parabolic lower portion (2) (2), which enters into the cavity,
a thin, elongated circular middle portion (3) (3), which is located between the lower portion (2) (2) and the surgical passages (4) and designed to increase air tightness,
3 surgical instrument insertion passages (4), which are located on the middle portion (3) and remain on the outermost part.

In the preferred embodiment of the invention, the lower portion (2) fits into the colpotomy line and is designed parabolically to reduce trauma during the placement process. The lower portion (2) is designed as shown in the “Figures” to provide the constant carbon dioxide pressure and air tightness necessary for the surgical technique. In the present invention, the lower portion (2) is obtained by rotating a hyperbolic curve 360 degrees around its axis and a circular opening is formed to allow the passage of instruments from the middle portion. In the present invention, this shape, which is also illustrated in detail in the Figures, is referred to as “parabolic”.

Thin middle portion (3) is formed by rotating the lower end of the curve in a wider circular plane, and includes 11 circular holes (6, 7) in total, namely 3 holes (7) for the passages (4) on the surface (position) of the passages (4) in contact with the middle portion (3) and 8 holes (6) for the passage of the suture thread. In the preferred embodiment of the present invention, the 3 holes (7) for the passages (4) are located on the central region of the circle and the 8 holes (6) for the passage of the suture thread are located on 4 positions of the circle, in groups of 2 on the sides of the circle, such that they are aligned on each of the 90 degree slices of the circle and the groups are equidistant from each other.

In the present invention, the passages (4) are hollow for allowing the passage of surgical instruments to the target area through the port (1) and the passages have cylindrical structure. On the surface of the passages (4) in contact with the middle portion (3), there are holes (7) with the same width as the hollow in the passages, and these holes (7), which are located on the middle portion (3), allow the surgical instruments coming from the passages (4) pass through the middle portion (3) and the opening on the lower portion (2). The passages (4) are placed on the 3 holes (7) of the middle portion (3) for the passages (4).

These passages (4) allow the endoscopic surgical instruments, which will be used during the surgical procedure, to be inserted into this port (1) and used in the procedure. The surgical instruments are inserted into the passages (4) located on the port (1) and this insertion process is performed by passing the surgical instruments, which will be used during the procedure, through the passages (4) located on the port (1) of the present invention, the holes (7) located on the middle portion (3) and then the opening (8) on the lower portion (2). Preferably, it has multiple passages (4) for the insertion of the different instruments, which will be used in the relevant surgical procedure.

The said 3 passages (4) are preferably designed for 1 endoscope (4.1) and 2 trocars (4.2) among the surgical instruments and the passages (4) allow these instruments to have access to the target tissue. There are three (3) passages (4), namely one passage of 10 mm (4.1) for the endoscope and the two other passages of 5 mm (4.2) for the other surgical instruments, preferably for the trocars. The said passages (4) are surgically ergonomic passages (4), which are positioned at an angle of 10 degrees to the vertical plane. Similarly, one of the holes (7) located on the middle portion (3) for the passages has a diameter of 10 mm and each of the other two has a diameter of 5 mm.

The present invention additionally comprises 3 air-tight, biocompatible, and elastic plugs (5), which can be attached to and detached from the location into which the surgical instruments first inserted, i.e., other aperture of the passages that is not in contact with the middle portion (3). One of these plugs (5) is a 10 mm plug and the other two are 5 mm plugs.

10 mm plug (5) is placed into the aperture, the outermost part of the cylindrical hollow in the passage of 10 mm. Prior to this process, a 6-7 mm aperture cut with the lancet on the surface where the plug will enter into the pathway is obtained and it is aimed to allow the endoscope to pass through this aperture. The two 5 mm plugs (5) are placed into the aperture, the outermost part of the cylindrical hollow in the passage of 5 mm. As is the case with the 10 mm plug, after creating 3-4 mm aperture cut with the scalpel on their surfaces, it is aimed to pass the surgical instruments therethrough and to continue the operation in an airtight manner. The presence of the plug (5) is for controlling the stability of the surgical instruments as well as acting as a valve which provides air tightness. Before the insertion of the surgical instruments into the passages, the said plugs (5) are fitted into the passages.

One of the elements in the present invention is the presence of 8 holes (6) provided on the middle portion (3) through which the 8 suture threads pass. By means of these holes (6), which are designed to increase the tightness and through which the suture threads will be passed, the suture threads are fixed between the patient's legs, creating a tension for the middle circular portion, and increasing the stabilization of the device.

In present invention, the said surgical instrument port (1) is manufactured with a biocompatible and elastic material suitable for the relevant surgical applications. The port is made of any biocompatible material which can be easily cleaned and sterilized by known methods according to the technical field.

In the preferred embodiment of the invention, the surgical instrument port (1) is disposable and manufactured specifically for the procedure and remanufactured each time. Therefore, it eliminates the need for sterilization between procedures.

Three-dimensional (3D) printing is a developing field due to the use of various novel materials and printing techniques. There are many fields where this technology can be used. One of these fields is the production of case-specific medical devices which are durable, high-quality, disposable, and cost-effective. A novel device, which is manufactured by 3D printing and serves as a surgical port, is designed for use in vNOTES. In a preferred embodiment of the invention, transvaginal surgical instrument port is obtained by being designed for 3D printing and manufactured with 3D printing application. In 3D printing application, the material used for manufacturing the invention is elastic biomaterial. Preferably, polylactic acid (PLA) is used as the material. Here, the invention is realized with a low-cost and ergonomic design by means of 3D printing.

Preferably, the port (1) of the present invention, which is manufactured by 3D printing, is disposable and can be produced specifically for the procedure. Therefore, it eliminates the need for sterilization between procedures.

The said invention can be manufactured by using standard techniques or production methods known in the state of the art.

With regard to these basic concepts, it is possible to develop a wide variety of applications by means of “Surgical Instrument Port for Vaginal Natural Orifice Transluminal Endoscopic Surgery” of the present invention and the invention, which is essentially as defined in the claims, cannot be limited to examples disclosed herein.

### REFERENCES

[1]. Yoshiki N. Review of transvaginal natural orifice transluminal endoscopic surgery in gynecology. Gynecol Minim Invasive Ther. 2017;6(1):1-5. Epub 20170120. doi: 10.1016/j.gmit.2016.11.007. PubMed PMID: 30254860; PubMed Central PMCID: PMC6113962.
[2]. Badiglian-Filho L, Fukazawa EM, Faloppa CC, Baiocchi G. VNOTES (Vaginal Natural Orifices Transluminal Endoscopic Surgery) myomectomy through anterior cul-de-sac approach on the bicornuate uterus. J Gynecol Obstet Hum Reprod. 2021;50(1):101911. Epub 20200912. doi: 10.1016/j.jogoh.2020.101911. PubMed PMID: 32931956.
[3]. Baekelandt J. Transvaginal natural-orifice transluminal endoscopic surgery: a new approach to myomectomy. Fertil Steril. 2018;109(1):179. Epub 20171109. doi: 10.1016/j.fertnstert.2017.09.009. PubMed PMID: 29129378.
[4]. Egi H, Okajima M, Hinoi T, Takakura Y, Kawaguchi Y, Shimomura M, et al. Single-incision laparoscopic colectomy using the Gelport system for early colon cancer. Scand J Surg. 2012;101(1):16-20. doi: 10.1177/145749691210100104. PubMed PMID: 22414463.
[5]. Feng D, He L. Regarding “Transvaginal natural orifice transluminal endoscopic surgery (vNOTES) in gynecologic surgeries: A systematic review”. Asian J Surg. 2020;43(8):842. Epub 20200504. doi: 10.1016/j.asjsur.2020.03.018. PubMed PMID: 32376215.
[6]. Wang Y, Deng L, Tang S, Dou Y, Yao Y, Li Y, et al. vNOTES Hysterectomy with Sentinel Lymph Node Mapping for Endometrial Cancer: Description of Technique and Perioperative Outcomes. J Minim Invasive Gynecol. 2021;28(6):1254-61. Epub 20210129. doi: 10.1016/j.jmig.2021.01.022. PubMed PMID: 33516958.
[7]. 7. Ozceltik G, Simsek D, Hortu I, Yeniel AO, Itil IM. Transvaginal natural orifice transluminal endoscopic surgery for ectopic pregnancy. J Obstet Gynaecol Res. 2022;48(3):843-9.
[8]. Alay I, Kaya C, Cengiz H, Yildiz S, Aslan O, Yasar L, et al. Apical pelvic organ prolapse repair via vaginal-assisted natural orifice transluminal endoscopic surgery: Initial experience from a tertiary care hospital. Asian J Endosc Surg. 2021;14(3):346-52.
[9]. Huang L, He L, Zhang L, Gan X, Jia J, Yang Y, et al. Application of the prone position in myomectomy by transvaginal natural orifice transluminal endoscopic surgery. Wideochir Inne Tech Maloinwazyjne. 2021;16(1):234-42.
[10]. Li CB, Hua KQ. Transvaginal natural orifice transluminal endoscopic surgery (vNOTES) in gynecologic surgeries: A systematic review. Asian J Surg. 2020;43(1):44-51.
[10]. Qin Z, Dong Z, Tang H, Zhang S, Wang H, Bao M, et al. A preliminary clinical report of transvaginal natural orifice transluminal endoscopic Sacrospinous Ligament Fixation in the treatment of moderate and severe pelvic organ prolapse. Front Surg. 2022;9:931691.

What is claimed is:

1. A transvaginal surgical instrument port for use in vaginal natural orifice transluminal endoscopic surgery, comprising:
a parabolic lower portion, wherein the parabolic lower portion comprises a circular opening and enters into a cavity,
a thin circular middle portion, wherein the thin circular middle portion is located between the parabolic lower portion and surgical passages and comprises 3 holes for the surgical passages on a surface of the surgical passages in contact with the thin circular middle portion and 8 holes for a passage of a suture thread, and
3 surgical instrument insertion passages with a hollow and cylindrical structure, wherein the 3 surgical instrument insertion passages are placed on the 3 holes of the thin circular middle portion for the surgical passages

2. The transvaginal surgical instrument port according to claim 1, wherein the 3 holes for the surgical passages are located in a central region of the thin circular middle portion.

3. The transvaginal surgical instrument port according to claim 2, wherein the 8 holes for the passage of the suture thread are located on 4 positions of a circle, in groups of 2 on sides of the circle, such that the 8 holes are aligned on each of 90 degree slices of the circle and the groups are equidistant from each other.

4. The transvaginal surgical instrument port according to claim 3, wherein the 3 surgical instrument insertion passages with the hollow and cylindrical structure comprise one passage with a width of 10 mm and two passages with a width of 5 mm.

5. The transvaginal surgical instrument port according to claim 4, wherein the surgical passages are positioned at an angle of 10 degrees to a vertical plane.

6. The transvaginal surgical instrument port according to claim 5, wherein one of the 3 holes for the surgical passages is located on the thin circular middle portion, and has a diameter of 10 mm, and each of the other two holes has a diameter of 5 mm.

7. The transvaginal surgical instrument port according to claim 6, wherein one of 3 surgical instruments is an endoscope and the other two are trocars.

8. The transvaginal surgical instrument port according to claim 1, further comprising 3 biocompatible and elastic plugs, wherein the 3 biocompatible and elastic plugs are configured to be attached to and detached from an aperture of the surgical passages, wherein the aperture of the surgical passages is not in contact with the thin circular middle portion.

9. The transvaginal surgical instrument port according to claim 8, wherein one of the 3 biocompatible and elastic plugs is a 10 mm plug and each of the other two is 5 mm plug.

10. The transvaginal surgical instrument port according to claim 8, wherein the 10 mm plug of the 3 biocompatible and elastic plugs comprises a 6-7 mm aperture cut with a first lancet on a first surface where the 10 mm plug will enter into a first pathway; and each one of the other 5 mm plugs comprises a 3-4 mm aperture cut with a second lancet on a second surface where the 5 mm plug will enter into a second pathway.

11. The transvaginal surgical instrument port according to claim 1, wherein the transvaginal surgical instrument port is made of any biocompatible and elastic material.

12. The transvaginal surgical instrument port according to claim 1, wherein the transvaginal surgical instrument port is manufactured by 3D printing.

13. The transvaginal surgical instrument port according to claim 2, further comprising 3 biocompatible and elastic plugs, wherein the 3 biocompatible and elastic plugs are configured to be attached to and detached from an aperture of the surgical passages, wherein the aperture of the surgical passages is not in contact with the thin circular middle portion.

14. The transvaginal surgical instrument port according to claim 3, further comprising 3 biocompatible and elastic plugs, wherein the 3 biocompatible and elastic plugs are configured to be attached to and detached from an aperture of the surgical passages, wherein the aperture of the surgical passages is not in contact with the thin circular middle portion.

15. The transvaginal surgical instrument port according to claim 4, further comprising 3 biocompatible and elastic plugs, wherein the 3 biocompatible and elastic plugs are configured to be attached to and detached from an aperture of the surgical passages, wherein the aperture of the surgical passages is not in contact with the thin circular middle portion.

16. The transvaginal surgical instrument port according to claim 5, further comprising 3 biocompatible and elastic plugs, wherein the 3 biocompatible and elastic plugs are configured to be attached to and detached from an aperture of the surgical passages, wherein the aperture of the surgical passages is not in contact with the thin circular middle portion.

17. The transvaginal surgical instrument port according to claim 6, further comprising 3 biocompatible and elastic plugs, wherein the 3 biocompatible and elastic plugs are configured to be attached to and detached from an aperture of the surgical passages, wherein the aperture of the surgical passages is not in contact with the thin circular middle portion.

18. The transvaginal surgical instrument port according to claim 7, further comprising 3 biocompatible and elastic plugs, wherein the 3 biocompatible and elastic plugs are configured to be attached to and detached from an aperture of the surgical passages, wherein the aperture of the surgical passages is not in contact with the thin circular middle portion.

19. The transvaginal surgical instrument port according to claim 2, wherein the transvaginal surgical instrument port is made of any biocompatible and elastic material.

20. The transvaginal surgical instrument port according to claim 3, wherein the transvaginal surgical instrument port is made of any biocompatible and elastic material.
